(19) United States
(12) Patent Application Publication
(10) Pub. No.: US 2009/0132015 A1
(43) Pub. Date: May 21, 2009

(54) Method and System for Using Directional Antennas in Medical Treatments

(76) Inventors: Mark Frazer Miller, Suwanee, GA (US); Delicia Lashaun Munfus, Birmingham, AL (US); John Williams Miller, Suwanee, GA (US); Jamel Lynch, Lynchburg, VA (US)

Correspondence Address: DARCELL WALKER, 8107 CARVEL LANE, HOUSTON, TX, 77036, US

(21) Appl. No.: 12/204,793

(22) Filed: Sep. 4, 2008

Related U.S. Application Data
(63) Continuation-in-part of application No. 11/986,126, filed on Nov. 20, 2007.
(60) Provisional application No. 60/967,317, filed on Sep. 4, 2007.

(51) Int. Cl. A61F 2/00

(52) U.S. Cl. 607/101

(57) ABSTRACT

A method and system uses heat generated by radio frequency (RF) signals induced hyperthermia to destroy abnormal cells that cause diseases. A patient's body is not invaded with any substance or equipment. This invention incorporates a physical phenomenon that occurs when RF signals are added. When the amplitudes of RF signals are added there is a marked increase in the amplitude of the resulting signal. The physics of RF signals causes the intensity of the resulting signal to quadruple. Heat is generated as a result of intensity. In the present invention, RF signals from multiple directional antennas are added at a target location. At this location, preferably the amplitudes of the RF signals that are in phase. When this occurs, the intensity at that target location dramatically increases thereby the heat at that point dramatically increases. The intense heat at the target location destroys the cells at the target location. However, the amplitudes of the RF signals are only added at the target location. As a result, the increased intensity and intense heat only occur at that target location. Therefore, the RF signals do not affect the body at any other location.

### CROSS-REFERENCE TO RELATED APPLICATIONS

This application is related to and claims priority from provisional patent application No. 60/967,317 filed on Sep. 4, 2007 and patent application Ser. No. 11/986,126 filed on Nov. 20, 2007, the contents of which are incorporated herein by reference.

### FIELD OF THE INVENTION

The present invention relates generally to the field of radio frequency (RF) signals, and more specifically to an RF system and method for using multiple radio frequency signals transmitted from directional antennas to generate hyperthermia at specifically identified target areas.

### BACKGROUND OF THE INVENTION

Many physical diseases, such as cancer, are caused when some cells become abnormal and begin to divide out of control. Cancer in particular can spread via the lymphatic and vascular systems to otherwise healthy tissue anywhere in the body. Current medical treatment for the various categories of cancer (e.g. brain, breast, liver, etc . . . ) can include one or more of the following treatments.

Chemotherapy which is a process that uses chemical substances to treat disease. In its modern-day use, it refers to cytotoxic drugs used to treat cancer or the combination of these drugs into a standardized treatment regimen. There are a number of strategies in the administration of chemotherapeutic drugs used today. Chemotherapy may be given with a curative intent or it may aim to prolong life or to palliate symptoms. Combined modality chemotherapy is the use of drugs with other cancer treatments, such as radiation therapy or surgery. Most cancers are now treated in this way. Combination chemotherapy is a similar practice which involves treating a patient with a number of different drugs simultaneously. The drugs differ in their mechanism and side effects. The biggest advantage of chemotherapy is minimising the chances of resistance developing to any one agent.

High-frequency Focused Ultrasound therapy has been used for thermal ablation of cancers. This minimally invasive method focuses ultrasound energy to heat up tissue without the need for an electrode or transducer. This method is however limited because air and bone can interfere with and limit ultrasound penetration. Consequently, only soft tissue tumors, near the skin surface can be targeted

Radiation therapy (or radiotherapy) is the medical use of ionizing radiation as part of cancer treatment to control malignant cells (not to be confused with radiology, the use of radiation in medical imaging and diagnosis). Radiation therapy is commonly applied to the tumour. The radiation fields may also include the draining lymph nodes if they are clinically or radiologically involved with tumour, or if there is thought to be a risk of subclinical malignant spread. It is necessary to include a margin of normal tissue around the tumour to allow for uncertainties in daily set-up and internal tumor motion. These uncertainties can be caused by internal movement (for example, respiration and bladder filling) and movement of external skin marks relative to the tumour position. To spare normal tissues (such as skin or organs which radiation must pass through in order to treat the tumour), shaped radiation beams are aimed from several angles of exposure to intersect at the tumour, providing a much larger absorbed dose there than in the surrounding, healthy tissue. Although radiation is an accepted form of cancer treatment, there are some side effects that accompany this method. There can be damage, possibly severe, to epithelial surfaces (skin, oral, pharyngeal and bowel mucosa, urothelium). Moreover radiation therapy can actually cause normal tissue to become cancerous.

Surgery is a medical specialty that uses operative manual and instrumental techniques on a patient to investigate and/or treat a pathological condition such as disease or injury, to help improve bodily function or appearance, or sometimes for some other reason. In some cases the cancer, particularly in metastatic brain cancer, can only be treated via surgery. Because of the position of some cancers, deep within the brain, they are virtually impossible to treat using today's surgical techniques.

Another form of radiation is RF electromagnetic radiation. It is known in the art to use to direct RF electromagnetic radiation to intentionally induce hyperthermia in human tissue for therapeutic purposes, e.g., destroying diseased cells. The theory that forms the basis for using radio frequency radiation is that when RF radiation is absorbed by matter it causes molecules to vibrate, which in turn causes heating. More specifically, RF waves interact with matter by causing molecules to oscillate with the electric field. This interaction has proven to be most effective for molecules that are polar, i.e. having their own internal electric field, such as water. Water molecules lose rotational energy via friction with other molecules, which causes an increase in temperature. This effect is the basis for microwave cooking. RF radiation absorbed by the body typically occurs as a result of the interaction of the RF radiation with water fluids contained in vivo.

The amount of RF radiation absorbed by tissue depends on a number of things, including the power and specific frequency of RF radiation used. Some frequencies of RF radiation have high absorption rates in tissue. A typical microwave oven emits RF radiation at about 2500 MHz, which is readily absorbed by water, fats and sugars to generate heat in food. RF radiation at lower frequencies, e.g., medium frequency (“MF”; 300 to 3000 kilohertz) RF radiation and high frequency (“HF”; 3 to 30 megahertz) RF radiation have generally low absorption rates in human tissue, even at relatively high powers, as evidenced by people safely standing near radio station tower transmitters, which transmit tens of thousands, and even hundreds of thousands, of Watts of RF power at lower frequencies.

RF ablation uses RF induced thermal energy to destroy tumor cells and involves placing a special needle into a tumor, often using image guidance. U.S. Pat. No. 4,800,899 discloses a system including a needle-like antenna that is inserted into a patient's body and into a tumor, permitting microwave RF energy supplied by a microwave generator to be applied directly to the tumor via the needle-like antenna to induce hyperthermia in the tumor. The RF energy generates heat in a volume (e.g., sphere) of tissue surrounding the needle. Ideally, the generated heat kills the tumor in a manner that spares the healthy tissue surrounding the tumor. RF ablation has several drawbacks, including the fact that treatment involves direct contact with the patient, i.e., insertion of a needle-like antenna into the patent for the duration of the procedure, which can require sedation and possibly an overnight stay in a hospital.

Another approach that uses RF waves to treat cancer is described in U.S. patent application publication 20050251233. This approach is a non-invasive RF system for inducing hyperthermia in a target area, and a corresponding non-invasive RF method for inducing hyperthermia in a target area. The system includes an RF transmitter and transmission head, and RF receiver and reception head wherein the transmission and reception heads are arranged proximate a target area so that an RF signal between the heads induces hyperthermia in the target area. The method includes arranging the transmission head and reception head proximate and on either side of a target area and transmitting an RF signal through the target area. The methodology further includes providing antibodies bound to an RF absorption enhancer and injecting the antibodies into the patient. Waiting for a period of time for the antibodies to bind to at least one type of cells within the target area and transmitting an RF signal from the transmission head to the reception head thereby warming the specific target portion of the target area of the body part.

Although this latest approach has received promising reviews, it still has some disadvantages. The method of inducing hyperthermia using only the RF transmitter and RF receiver components does not induce sufficient hyperthermia to destroy a tumor at a target area. The waves that are transmitted must be at a frequency that will generate heat but no so much heat that the waves will harm the patient. To increase the heat at the target area, this method bounds antibodies with RF absorption enhancers. This combination of antibodies and RF absorption enhances is injected into the patient. At this point, this method becomes invasive. The antibodies and RF absorption enhancers bind to the target area and begin to absorb RF signals at that point. The absorption enhancers enable substantial heat to build up at the specific location of the absorption enhancer. The heat build up at the RF absorption enhancers eventually destroys the target. However, the heat build up in the rest of the patient is only caused solely by the RF signals and is does not generate enough heat to be harmful to the patient. Another challenge of this method is to get the RF absorption enhancers to locate and accurately and correctly attach to the defined target area. Lastly, since antibodies are used in this method, retargeting the same type of tumor in the same patient will be difficult. The human body naturally makes antibodies to fight foreign antibodies introduced to the body, such as what is used in the above method. Consequently, the first set of antibodies introduced to the body attached to these RF absorption enhancers may be effective, since the body will take several days to make antibodies to fight the foreign antibodies. However, the human immune system has a great memory and will get rid of a second set of the same antibodies before they can target the tumor, which will hinder treating the same patient for the same type of tumor. This limitation is not present in our RF therapy method.

Another approach for using RF signals in medical treatments is to continuously but intermittently fire RF signals at a target area. This process intends to fire enough RF signals at the target such that heat from the RF signals will began to accumulate at the target location and destroy the target. One major disadvantage with this approach is that the RF firing is intermittent thereby requiring substantial time to accumulate enough heat to affect the target area. In addition, continuous RF firing could create cause damage to healthy cells and body tissue along the path that the RF signal travels.

Dr. Marie Curie, two time Nobel Laureate, had a brilliant/cross disciplinary idea of using radiation from radium to kill cancer. Thinking about and looking closer at what Dr. Curie observed and advanced in cancer treatment was that the wavelength of the radiation given off by radium disrupted the chemical bonds of molecule within the cell, which eventually killed them. This physics based approach to treating cancer is now being studied and use in Hospitals and Research institutes worldwide. From her research was spawn an important and powerful brain cancer fight instrument called the Gamma-Knife. The Gamma Knife is a $3.5 million, 20-ton tool that is used to performs Stereotactic Radiosurgery using a concentrated cobalt radiation dose delivered with precision to destroy abnormal issues without an incision or damage to surrounding normal tissue. After treatment, most of the patients (85%) are cure within 2 years. Unfortunately, this treatment is not available to the many people who need it.

The medical research seeking to find cures for multiple diseases and in particular cancer has produced innovative approaches to treating diseases. However, traditional approaches to fighting cancer, up until now, have involved creation of drugs and crude chemotherapy and radiation treatments that kill not only the cancer cell but also important healthy cells. The present methods for treating diseases such as cancer damage healthy human cells as a consequence of attacking the abnormal cells. When healthy cells are damaged, the possibility of weakening the body and subsequent infection increases. When the body is weaken the possibility of making the disease worse increases. In addition, the primary treatment techniques also involve invading the human body. The invasions can be as simple as ingesting a drug or as drastic as surgery. Further, as well known some cancer tumours are located in the human body in places where an attempt to treat them with any form of invasion technique will result in the patient's death. These types of cancers are described as inoperable. However, there still remains need for a medical treatment method and system that can efficiently destroy abnormal human cells without harming healthy tissue adjacent the abnormal cells or cells anywhere else in the body. Further there is a need for medical techniques that can treat diseases without invading the body.

### SUMMARY OF THE INVENTION

A method and system of the present invention uses heat generated by radio frequency (RF) signals to destroy abnormal cells that cause diseases. This method incorporates a physical phenomenon that occurs when RF signals are added. When the amplitudes of RF signals are added there is a marked increase in the amplitude of the resulting signal. The physics of RF signals causes the intensity of the resulting signal to quadruple. Heat is generated as a result of intensity. In the present invention, multiple RF signals are added at a target location. At this location, preferably the amplitudes of the RF signals that are in phase. When this occurs, the intensity at that target location dramatically increases thereby the heat at that point dramatically increases. The intense heat at the target location destroys the cells at the target location. However, in this invention, the amplitudes of the RF signals are only added at the target location. As a result, the increased intensity and intense heat only occur at that target location. Therefore, the RF signals do not affect the body at any other location.

In the method of the present invention, a target is identified. This target is one or more abnormal cells in the human body. A cancerous tumour is an example of a group of abnormal cells that would be defined as a target. Once there is an identification of a target, the method next determines the location or position of the target. The location of the target will be used to determine the orientation and positioning of RF emitters. The target location can be determined as a physical location in space having appropriate coordinates to identify this physical location. After determining the target location, RF emitters are oriented such that a signal emitted from an RF emitter will travel through the target location. In the present invention two or more RF emitters will transmit RF signals through the target location. In the embodiment of the present invention, directional antennas serve as the emitters or source of the RF signals. This heat is sufficient to destroy the cancer cells. The RF emitters transmit RF signals such that each signal travels through the target but also the signals coalesce at the target location. When the signals coalesce, the amplitudes of the signals add or sum to produce increased amplitude that is the total of the individual signal amplitudes.

### DESCRIPTION OF THE DRAWINGS

FIG. 1 is a graphic representation of a uniform radio frequency (RF) waveform.

FIG. 2 is a graphic representation of two uniform radio frequency (RF) waveforms that are in phase.

FIG. 3 is a graphic representation of a resulting radio frequency (RF) waveform that results from adding the two RF waveforms in FIG. 2.

FIG. 4 is an illustration of the implementation of the method and system of the present invention to a brain tumor using directional antennas to supply the radio frequency (RF) signals in accordance with embodiments of the present invention.

FIG. 5 is a graphic representation of a radio frequency (RF) waveform illustration a complete cycle of the waveform.

FIG. 6 is a graphic representation of multiple radio frequency (RF) waveforms representing RF signals that can be transmitted into a target location in accordance the method and system of the present invention.

FIG. 7 is an illustration of a configuration of the system of the present invention.

FIG. 8 is an illustration of the present invention using sets of concentric rings, small DSP processors (RF-generators) with wave-guides, and precise computer positioning controls.

FIG. 9 is a configuration of the implementation of the system of the present invention using a parabolic RF signal reflector.

FIG. 10 is a flow diagram illustrating the basic steps in the implementation of the method of the present invention.

FIG. 11 is a detailed description of an embodiment of the present invention.

FIG. 12 is a more detailed flow diagram illustrating the steps in an implementation of the method of the present invention.

### DETAILED DESCRIPTION OF THE INVENTION

The present invention provides a non-invasive method and system for treating diseases. This system is truly non-invasive in that no part of the method for implementing this invention will require any foreign object to enter the body at any time as part of the treatment or to facilitate the implementation of the method. This present invention uses heat generated by radio frequency signals to apply heat to an identified target area and destroy abnormal cells at that target area in a single treatment. Although heat is generated and applied to an identified target area, effectively no heat is applied to area outside the identified target area. This ability to only apply heat to a specific area enables the present invention to transmit RF signals through a human body without affecting the person.

In the implementation of the present invention, the RF signals generate heat at a specific location similar to conventional medical treatments that use RF signals. However, the present invention relies on a phenomenon in physics referred to as “coalescing in phase wave theory using RF” to instantaneously generate intense heat at a defined target location instead of relying on a conventional approach of heat build up via delivery of a waveguide to deliver RF frequencies at the specific location. The present invention uses a term coined called “thermal singularity” to generate instantaneous heat at a specified target location. In thermal singularity, multiple RF signals are emitted at the same frequency and from different directions traveling from different paths through the same target location. When these signals coalesce (intersection), they produce an instantaneous heat build up at that target location that destroys the target. This concept of thermal singularity has its' basis in the physics of wave theory applied to RF waves. Below is an explanation of the concept of thermal singularity with regard to RF signals.

### RF Thermal Singularity

The basic physics concept of RF signals, which supports the approach of the present invention, is as follows. A radio transmitter emits an RF signal uniformly with an amplitude A, frequency F, power P and intensity I. When a second identical transmitter, that has the same frequency in phase with the first signal, is added to the first signal, the amplitudes of the two waves add resulting in double the amplitude of the signals. However, the adding of the signals also results in an increase in the intensity of the RF signal by 4 times the initial intensity of a signal.

RF Signal Amplitude

RF signals have amplitude, which is the objective measurement of the degree of change (positive or negative) in atmospheric pressure (the compression and rarefaction of air molecules) caused by sound waves. Sounds with greater amplitude will produce greater changes in atmospheric pressure from high pressure to low pressure. Amplitude is almost always a comparative measurement, since at the lowest-amplitude end (silence), some air molecules are always in motion and at the highest end, the amount of compression and rarefaction though finite, is extreme. In electronic circuits, expanding the degree of change in an oscillating electrical current may increase amplitude. Amplitude is directly related to the acoustic energy or intensity of a sound. Both amplitude and intensity are related to sound's power. All three of these characteristics have their own related standardized measurements. Amplitude is measured in the amount of force applied over an area. The most common unit of measurement of force applied to an area for acoustic study is the Newton's per square meter (N/m²). Discussions of amplitude depend largely on measurements of the oscillations in barometric pressure from one extreme (or peak) to the other. The degree of change above or below and imaginary center value is referred to as the peak amplitude or peak deviation of that waveform.

RF Signal Power and Intensity

A sound wave as an expanding sphere of energy, power is the total amount of kinetic energy contained on the sphere's surface. The below formula illustrates how power is a measurement of amplitude over time.

1 watt=1 Newton of work per second

The unit of measurement for power is the watt. The power of the original sound source and the distance of measurement from the original sound source to the target area combine to form the intensity. Intensity can be measured as watts per square meter or w/m². Intensity can be seen as amplitude over time over an area. As the surface area of the sound sphere expands, the amount of energy generated by the sound source is distributed over an exponentially increasing surface area. The amount of energy in any given square meter of the expanding sphere's surface decreases exponentially by the inverse square law, which states that the energy drops off by 1/distance². Therefore, acoustic energy twice the distance from the source is spread over four times the area and therefore has one-fourth the intensity, or simply put, relative intensity is the reciprocal of the change in distance squared. Intensity equals the square of the amplitude, so if the amplitude of a sound is doubled, its intensity is quadrupled. Power and intensity are proportional to each other.

These relationships are based on the phase of signals. The power is most directly affected by phase, namely the phase of the waves as they arrive at the target location. The primary phase possibilities are:
1 & 1 makes 4 (which is power at 0° phase shaft)
1 & 1 makes 0 (which is power at 180° phase shift)
1 & 1 makes 2 (which is power at 90° phase shift)
   The present invention is based on the first listed possibility where the two amplitudes produce a quadrupling (4 times) the power.

RF Signals

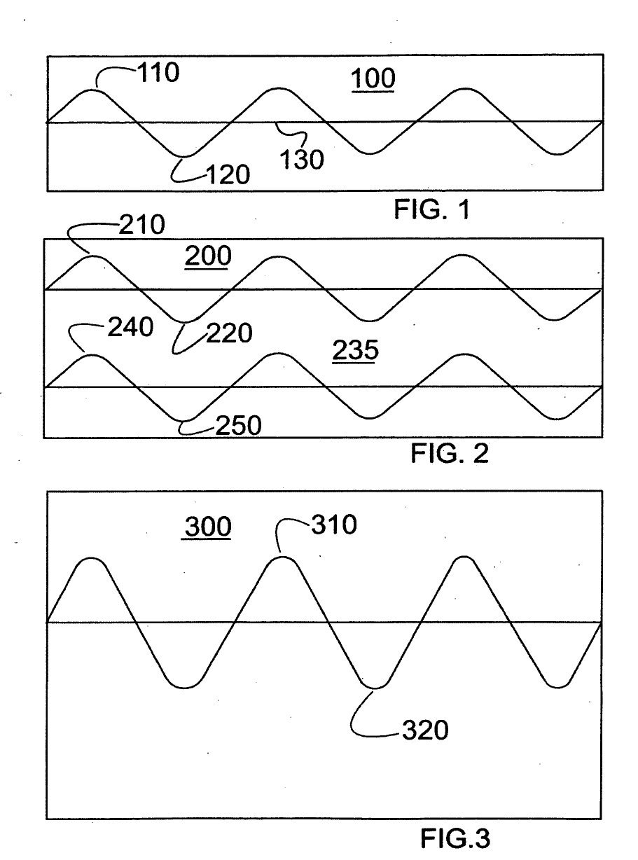

FIG. 1 shows a conventional uniform sinusoidal waveform that will be used to explain the implementation of the RF signals in the present invention. The waveform 100 has positive 110 and negative 120 peak amplitudes. The amplitude is the distance of a particular point on the waveform from reference zero 130. The waveform propagates in repetitive cycles called periods. The number of cycles over a defined period of time is known as the frequency of that waveform. Frequency is the measurement of the number of occurrences of a repeated event per unit of time. It is also defined as the rate of change of phase of a sinusoidal waveform. Frequency has an inverse relationship to the concept of wavelength. The frequency f is equal to the speed v of the wave divided by the wavelength λ (lambda) of the wave:

f=v/λ

In the special case of electromagnetic waves moving through a vacuum, then v=c, where c is the speed of light in a vacuum, and this expression becomes:

f=c/λ

When waves travel from one medium to another, their frequency remains exactly the Same—only their wavelength and speed change. The present invention uses a lower frequency which is a signal with a longer wavelength.

FIG. 2 shows two sinusoidal waveforms similar to FIG. 1. When the amplitude of the waveforms are combined they produce a resultant wave that has an amplitude that is the sum of the amplitudes of the individual waveforms that were added. In FIG. 2, the peak amplitudes of both wave forms is 2. Since the waveforms are in phase each point on the waveforms will add and produce a greater resultant amplitude. If the waveforms are not phase, amplitude values at various points will subtract resulting in a decreased amplitude at that point. In this example the waveforms are added at the positive peek amplitudes 210 and 240 and the negative peek amplitudes 220 and 250. FIG. 3 shows the result waveform 300 from the sum of waveforms 200 and 235. In this waveform 300, the positive peek amplitudes 310 and the negative peek amplitudes 320 have a value of 4 which is the sum of the amplitudes of the two waveforms. Each waveform had a value of 2. However, when the signals are in phase, the intensity of the wave in Figure is quadruple the value of an initial wave.

### Implementation of the Present Invention

The present invention has many medical applications. The method and system can be used to identify and destroy many types of abnormal cells. One primary use of the present invention is in the treatment of cancerous tumors. Other types of tumors can also be treated and destroyed with this invention. Tumors such as fibroid tumors can be removed without invading the body. With the elimination of the physical invasion, recovery time for a disease is substantially reduced and eliminated in some cases.

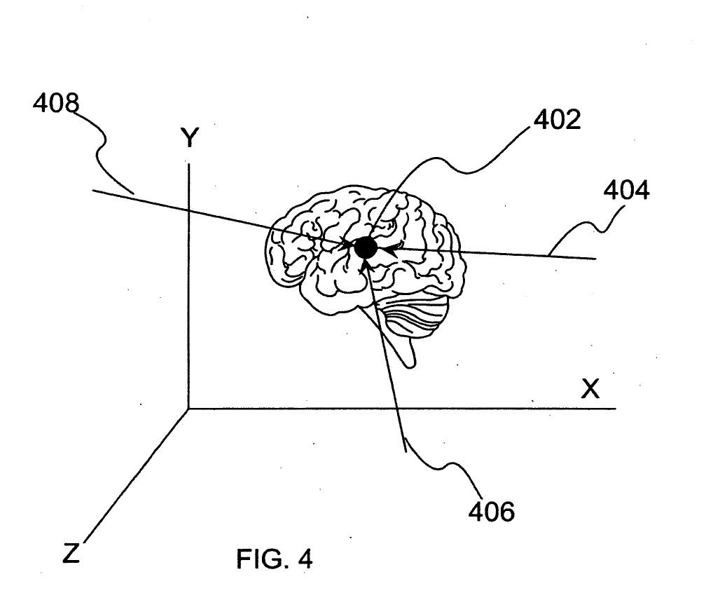

An implementation of the present invention illustrated in FIG. 4 is for treatment of a brain tumor using directional antennas to supply the radio frequency signals. The present invention uses multiple high gain directional antennas (i.e. Yagi directional antennas) develop a technique to focus the RF energy for transmission within a micron of the target (matastatic brain cancer). Using existing imaging techniques, metastatic brain cancers can be imaged and its position exactly calculated to within 1 micron. Using the appropriate number of Yagi antennas an “RF thermal singularity” resulting from individual sine waves will coalesce and their amplitudes (i.e. Power) will add at the intersection point of the independent RF sine waves. The singularity can/will deliver thermal heat (The RF thermal singularity) at that point for a controllable amount of time (e.g. Milliseconds, seconds, minutes, etc . . . ).

FIG. 4 illustrates the concept of using the multiple directional antennas to generate thermal energy at the point of an identified tumor in order to destroy (burn up) the tumor. This implementation is based on the concept of antenna gain. Shown are three directional antennas with the capability to direct RF signals through a target location (tumor) in the brain. Each antenna emits an RF signal in a direction that will cause to signal to pass through an identified tumor location. The RF signals from the emitters will be directed such that these signals intersect or coalesce at the location of the tumor. At this location, the thermal energy generated by the combined RF signals will be able to destroy the tumor. In this process, thermal energy generation will only occur at the point that the RF signals intersect (which is the location of the tumor). The individual RF signals passing through the body will not have any effect on the person except at the point where the signal intersect.

The use of directional antennas in the implementation of the present invention requires the determination of multiple issues. One key to successfully using Yagi antennas to remove the tumor (i.e. matastatic brain cancer) is the correctly orientate and align transmitting antennas. This involves determining how to aim and position the additional elements (reflector/director) placed in front of the antenna to focus the energy for transmission within a micron of the target (increase strength and narrow the direction of the signal prior to transmission), followed by researching the appropriate signal strength and duration required to remove the matastatic brain cancer are area of investigation.

A second issue in the implementation of directional antennas in this invention is the concept of antenna “gain” or signal intensity. The intent is to focus the radiated energy of the transmitter within 1 micron of the target. The transmitting power and properly aligning multiple antennas are key elements to identify the threshold transmit power required to remove the cancer. Water, fats and sugars absorb radio waves in the 2.5 gigaherz frequency range. When they are absorbed they are converted directly into atomic motion, which is heat. The fusion of the Radio Frequency sine waves, that become one very large wave at the singularity is what we are coining the RF-Knife!

As mentioned, the method of the present invention involves the use directional antennas to treat cancer by identifying and destroying cancer cells. This invention involves identifying the location of a target (a cancer cell). The antennas are positioned at known locations with regard to the target location. When the target is stationary, this identification can include defining space within which the target is located. With the location of the target known, there can be a determination of the orientation of each antenna such that an emitted RF signal would pass through the target. The emissions of the RF signals from each antenna are generally linear. As a result, if each signal passes through the target location, this location will be the point of intersection for the RF signals. The point of intersection is the point of the generated heat sufficient to destroy the target.

In this method, in addition to determining the orientation, it is also necessary to determine signal precision, signal intensity (strength) and signal duration. Signal precision relates to the width (wide or narrow) of the signal. This precision will be based on the size of the target. For larger targets, the signal will have a wider precision. For smaller targets, the precision could be narrower. The signal precision can influence the signal intensity. A wider signal precision may require an increased intensity (signal strength). The signal intensity can influence the signal duration. The higher the signal intensity, the shorter the signal duration can be to destroy the target. Different strategies can be implemented with regard to how to manipulate the signal precision, intensity and duration. However, the implementation of the signal precision, intensity and duration illustrate the need to characterize the target. This characterization would include information about the target dimensions.

Referring to FIG. 4, this image is one of a human brain. The tumor 402 is located in the center of the brain. This location is called the target location with the brain tumor being the target. Conventional procedures to treat this tumor require an invasion into the brain. This process is very risky and dangerous. In some instances, an attempt to perform surgery to remove the tumor is not possible without a major risk of harm to the person. These types of tumors are described as inoperable. The risks are so great, the physicians will not attempt to treat the patient. The present invention has the potential to eliminate so called inoperable cancers.

In the present invention, multiple radio waves are aimed at and transmitted to the abnormal cells. As shown, the three RF signals 404, 406 and 408 converge at the target tumor. As previously discussed, the RF signals will coalesce or intersect at the brain tumor. The amplitudes will add and the intensity and resulting heat generated by the RF signals will be absorbed at the tumor. The physic of adding of RF signals will cause quadrupling of the intensity of the signals and an instantaneous generation of heat. This heat will destroy the tumor. Natural body processes will remove the dead tumor cells. As shown, the RF signals come from different directions, which provides only for adding amplitudes at the point that the signals intersect at the target. Therefore, the remainder of the body tissue around but not part of the target area is not affected by the propagating RF signals.

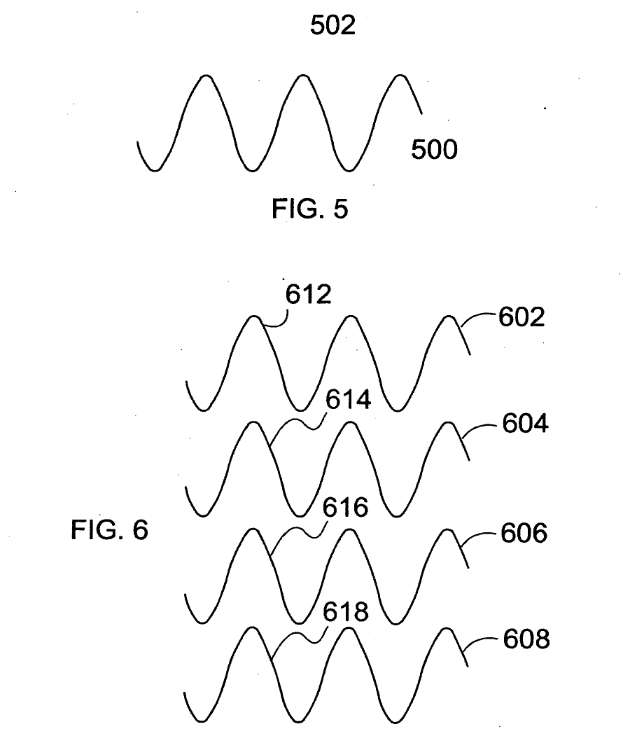

FIG. 5 is a graphic representation of a radio frequency (RF) waveform 500 illustrating a complete cycle of the waveform 502. As previously mentioned, preferably, the amplitudes of the signals should be in phase for the amplitudes to add and increase in value. At the point of the intersection of the signals, each signal must be at approximately the same point in the waveform cycle in order for the signal amplitudes to sum. Being at approximately the same point on the waveform cycle is illustrated in FIG. 6.

As shown FIG. 6 is a graphic representation of multiple radio frequency (RF) wave forms representing RF signals that can be transmitted into a target location in accordance the method and system of the present invention. These RF signals 602, 604, 606, and 608 are all sinusoidal waves. The present invention will to have each RF signal intersect the target location at the same point on the waveform. In FIG. 6, each waveform has an identified point: waveform 602 has point 612, 604 has point 614, 606 has point 616, and 608 has point 618. When these signals coalesce at the target location, it is desired for each wave to be at the identified point of that wave. These points represent a point on each wave when the amplitude is near the top, but slightly decreasing. If one or more waves were out of phase such as one being near the bottom of the wave, then the values could cancel, which would result in little to no resulting amplitude increase. As a result, there would be little intensity and thus little generated heat at the target location.

The concept of phase margin (pm) allows for some variance or phase in the lining up of the signal points. The points do not need to be 100% in phase for adding of the amplitudes to occur. Therefore, a range of amplitudes is available on each RF signal to get the desired coalescing and amplitude increase. For example, amplitude values of 1.8, 1.85, 1.9, 1.93 and 1.88 are not identical but are within the phase margin and are in phase. The desired physical result of the adding of the amplitudes is still achieved although the RF signals are not at the same point when intersecting at the target area. However, as previously shown, the greater the phase shift of the signals, the smaller the amplitude increase.

In summary, to a good approximation, with two RF signals, the present method and system can produce four times as much energy as with one separate RF signal. This is twice as much as one would predict by simply adding the power levels. Note, however that everything is valid “to a good approximation” but not exactly. This approximation is because amplitudes may not be exact. As previously discussed, the amplitude contributed by one signal will be slightly greater than 1, while the amplitude contributed by another will be slightly less than one. This does not appreciably affect the main results. In fact, there is a whole set of points where “extra” power can be produced, according to this concept.

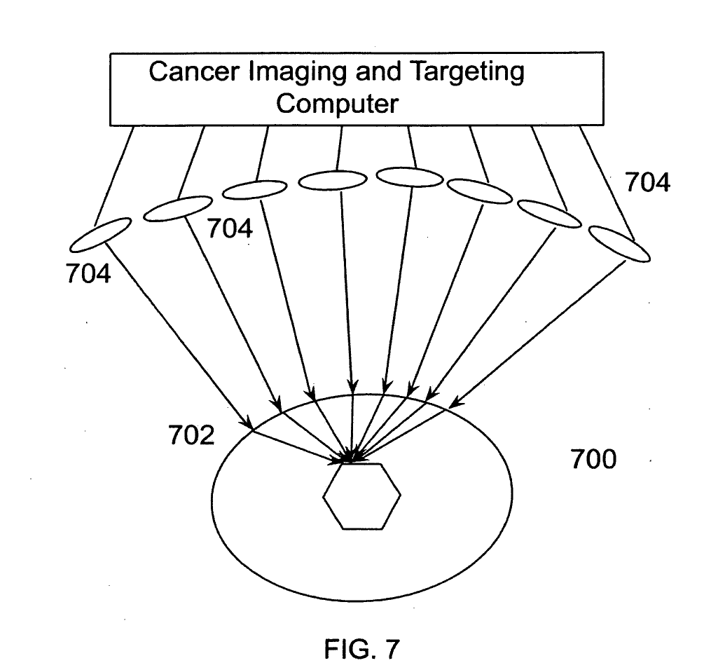

FIG. 7 is an illustration of a configuration of the system of the present invention. Shown is an abnormal cell (such as a cancerous tumor) 700 to which the system of the present invention can apply. This system comprises multiple RF emitters 704 that transmit RF signals 702 to the abnormal cell. An imaging and targeting computer 706 control these RF emitters. This computer can contain imaging software and capabilities to accurately identify abnormal cells. The imaging equipment can have MRI capabilities. Based on measurements performed after the identification of the target, this computer has software with the capability to determine the physical location in 3-Dimensional space of the abnormal cells. Once this location is determined, software will orient each RF emitter such that RF signal are emitted directed toward the identified target. In one embodiment, there can a calculation of how to fire the emitters such signals from each emitter arrive at the target location in phase to create the instantaneous heat effect at the target area T

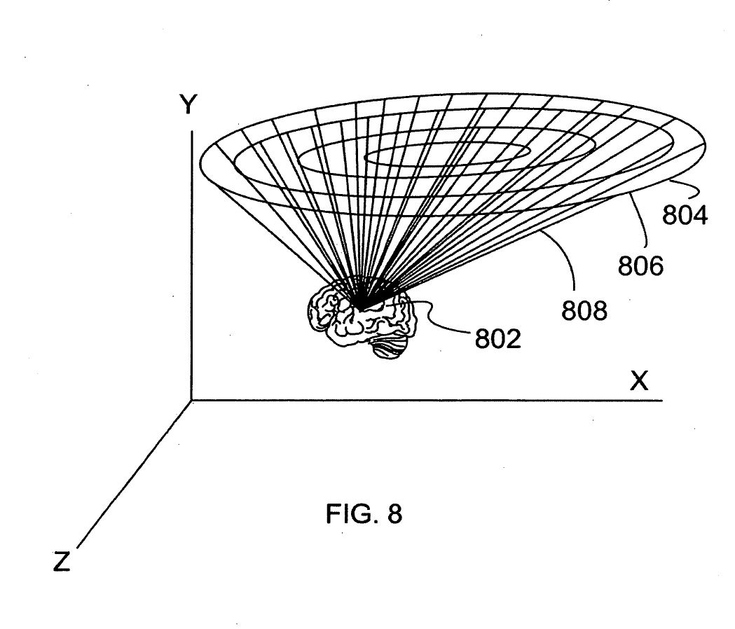

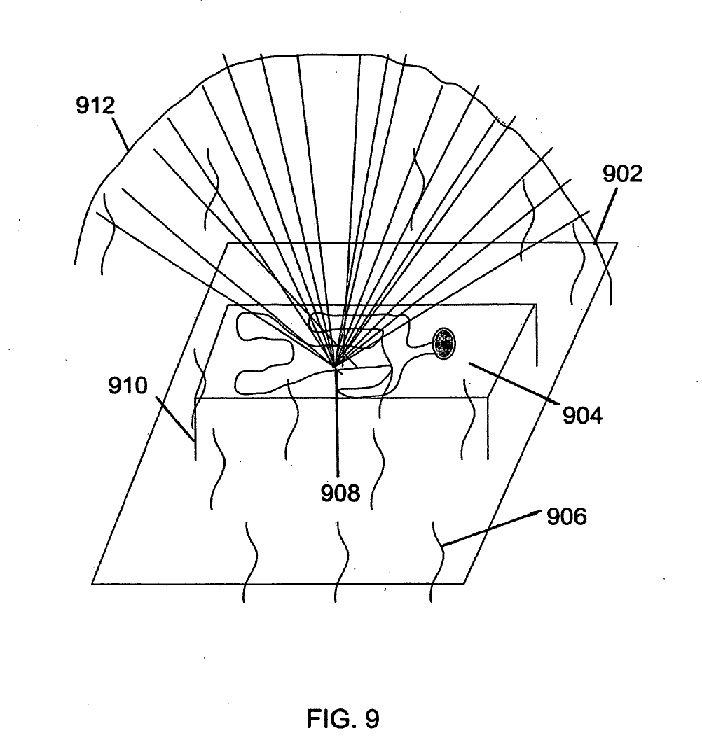

FIG. 8 is an illustration of an embodiment of the present invention. As shown, 802 represents a “traditionally” in-operable brain tumor. This embodiment uses sets of concentric rings, 804 and small DSP processors 806 (RF-generators) with wave-guides for focus RF signals 808 on the tumor. Computer controls are used to precisely position the RF generators. In addition, using MRI imaging and the data used for precise positioning, the physical location of the tumor in the brain can be located. Note that the present case is a brain tumor, however, a number of other targets like blockages in an artery can be targeted. Using the positional information (i.e. the physical location), the master-targeting computer could adjust the positions or orientations of the DSP RF-emitters 804 to strike at the position of the target indicated or displayed. A surgeon or other personnel would fire the emitters. The signals from the emitters would travel to the location of the tumor. When the signals intersect and coalesce a thermal singularity would be created at the position of the tumor.

FIG. 9 is a configuration of an implementation of the system of the present invention using a parabolic RF signal reflector. In this configuration, there is a uniform plane 902. The patient 904 has abnormal cells that form the target area 908. The patient lies on a table 910. This table could assist in establishing a physical location of the target area 908. RF emitters located below the patient can emit RF waves that travel up through the patient and reflect off of the parabolic reflector 912. The parabolic reflector can be positioned such that the RF waves strike the reflector from different directions but are all reflected back through the target area as the waves travel through the target area approximately in phase, heat is generated that will destroy the target area or abnormal cells.

Another configuration of an implementation of the present invention is to have multiple RF emitters aligning the edge of the table 910 in FIG. 9. The emitters could have known positions along the table or laying surface. Once the target area has been identified, the emitters could be positioned to emit RF waves through the target area and cause the destruction of the abnormal cells in the target area. In addition, directional antennas can be used in a system configuration to transmit RF waves through the target area of a patient.

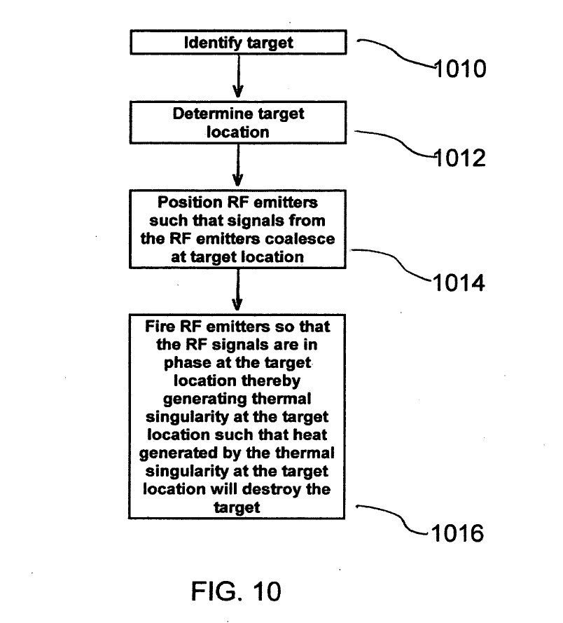

FIG. 10 illustrates a flow diagram of the general steps in the implementation of the method of the present invention. The initial step in the method is to identify the abnormal cells in the body 1010. One approach to accomplish this task is to visualize and delineate the cancer or abnormal cells using a radiolabeling technique such as Chlorotoxin or contrast agents such as Gadolinium. Experiments have shown that it is possible to illuminate brain cancer cells for better visualization and more importantly better targeting. Once there is an identification of a target, the method next determines the location or position of the target. The location of the target will be used to determine the orientation and positioning of RF emitters. The target location can be determined as a physical location in space having appropriate coordinates to identify this physical location. After determining the target location, in step 1012, step 1014 orients the RF emitters such that a signal emitted from an RF emitter will travel through the target location. In the present invention two or more RF emitters will transmit RF signals through the target location. The RF emitters transmit RF signals such that each signal travels through the target but also the signals coalesce at the target location. Step 1016, fires RF emitters so that the RF signals are in phase or approximately in phase at the target location thereby generating thermal singularity at the target location such that heat generated by the thermal singularity at the target location will destroy the target.

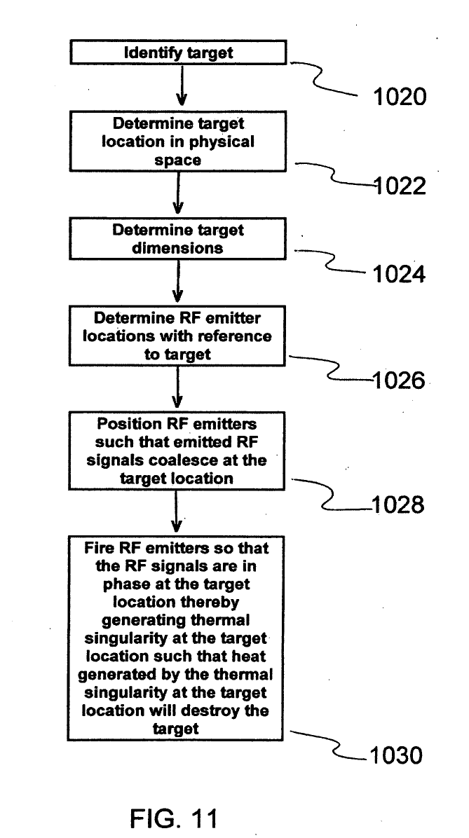

FIG. 11 is a detailed flow diagram illustrating the steps in an implementation of the method of the present invention. As with FIG. 10, step 1020 is to identify the target, which are abnormal cells such as cancerous tumors. Step 1022 determines a physical location of the target. This location could be a physical location in the body or a location in space. Step 1024 determines the physical dimensions of the target. It is helpful to know the size and shape of the target. This can be accomplished using medical imaging equipment like MRIs or CT scanning. This physical dimensions determination step can be optional. Step 1026 determines the RF emitter locations with reference to the target location. Since each emitter is in a physically different location, the orientation of each emitter is different. Depending on the type of target location scheme, this step may not be necessary. Once there is a physical relationship between the target area and an emitter, step 1028 positions or orients the RF emitters such that emitted RF signals travel through the body and coalesce at the target location. Step 1030, fires RF emitters so that the RF signals are in phase or approximately in phase at the target location thereby generating thermal singularity at the target location such that heat generated by the thermal singularity at the target location will destroy the target.

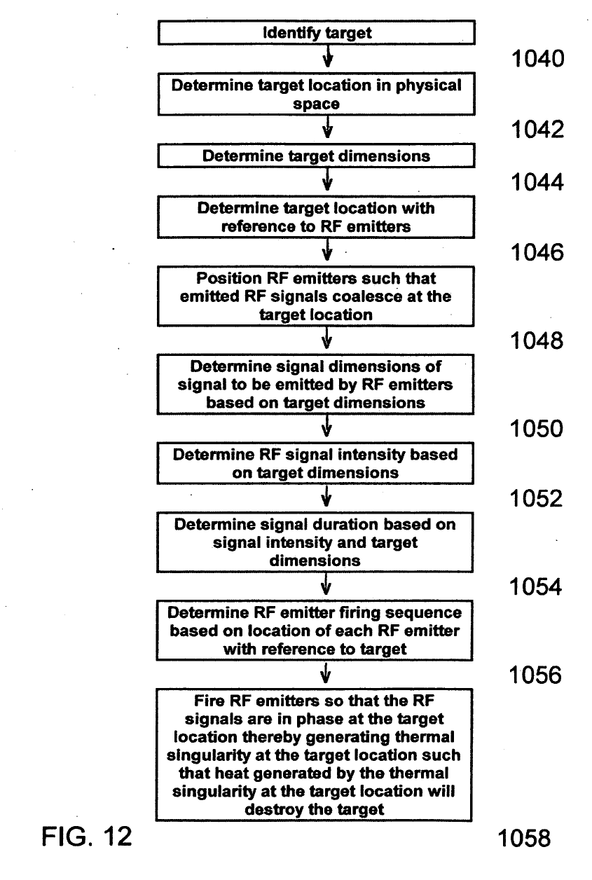

FIG. 12 is a more detailed flow diagram of the methods of FIGS. 10 and 11. In this method steps 1040, 1042, 1044, 1046 and 1048 and the same steps respectively as steps 1020, 1022, 1024, 1026, 1028 in FIG. 11. In this method, in addition to determining the orientation, it also may be desirable to determine signal precision, signal strength and signal duration. Signal precision relates to the width (wide or narrow) of the signal. This precision will be based on the size of the target. For larger targets, the signal will have a wider precision. For smaller targets, the precision could be narrower. The signal precision can influence the signal strength. A wider signal precision may require increased signal strength. The signal strength can influence the signal duration. The higher the signal strength, the shorter the signal duration needed to destroy the target. Different strategies can be implemented with regard to how to manipulate the signal precision, strength and duration. However, the implementation of the signal precision, strength and duration illustrate the need to characterize the target. This characterization would include information about the target dimensions. Step 1050 determines the signal dimensions for a signal that will be transmitted by an RF emitter to the target area. Step 1052 determines signal strength. Step 1054 determines signal duration based on the signal strength and target dimensions. Depending on the particular implantation of the method of the invention, steps 1050, 1052 and 1054 may be optional. Step 1056 determines an RF emitter firing sequence for the RF emitters based on the locations of the RF emitters with reference to the target. This step also may be option based on the particular implementation of the method of the present invention. As with FIG. 10, step 1058, fires RF emitters so that the RF signals are in phase at the target location thereby generating thermal singularity at the target location such that heat generated by the thermal singularity at the target location will destroy the target.

As mentioned, one particular application of the present invention is to treat brain tumors. Currently, less than 20% of brain cancers can be completely resected. A specialized processor will be created to interface to existing imaging equipment but will use custom algorithms (the physics of not only imaging and measurement but also targeting!) to adjust and control multiple RF emitters. The RF signals will coalesce the “rapid-fire” short duration multiple beams at the illuminated and precisely targeted and destroy brain cancer cell(s). The multi-disciplinary design of the present invention will provide a “safe”, economical, custom multi-gate array I/O optimized processor controlled automated cancer targeting system that can destroy brain cancer cells that cannot be reached using today's traditional manual surgical techniques. After successful and reliable destruction of brain cancer cells, the system will be adapted to attack other cancers, cells, bacterias and viruses, etc . . . that can be imaged and targeted for destruction.

While the present invention has been illustrated by the description of embodiments thereof, and while the embodiments have been described in some detail, it is not the intention of the applicant to restrict or in any way limit the scope of the appended claims to such detail. Additional advantages and modifications will readily appear to those skilled in the art. The invention in its broader aspects is not limited to the specific details, representative apparatus and methods, and illustrative examples shown and described. Accordingly, departures may be made from such details without departing from the spirit or scope of the applicant's general inventive concept.

We claim:

1. A system for using radio frequency signals to treat medical diseases comprising:
an element that identifies one or more abnormal cells and established the identified cell(s) as a target, said identifying element also determines a location of the target;
at least two RF emitters that transmit radio frequency signals, said RF emitters being directional antennas; and
a positioning module in communication with the RF emitters and the location determining element for receiving target location information from the location determining element and for positioning the RF emitters such that the RF emitters transmit radio frequency signals through the identified target in a manner such that thermal singularity is achieved at the target location.

2. The system as described in claim 1 further comprising a central controller for controlling operations of the RF emitters and the positioning module, said positioning module being contained in the central controller.

3. The system as described in claim 1 further comprising a firing module for firing the RF emitters at the target location at various firing sequences.

4. The system as described in claim 1 wherein said abnormal cell identifying element comprises a magnetic resonance imaging function.

5. The system as described in claim 1 wherein said abnormal cell identifying element comprises a computed tomography scanner function.

6. The system as described in claim 2 wherein said controller further comprises a signal adjuster module that can vary characteristics of an RF signal transmitted by an RF emitter.

7. A method for using radio frequency signals to treat medical diseases comprising the steps of:
determining a location of an identified target, the target being one or more abnormal cells;
orienting at least two directional antenna RF emitters that are capable of transmitting an RF signal such that a signal transmitted from an emitter passes through the identified target location; and
firing a directional antenna RF emitter so that a signal transmitted from an RF emitter will travel through the target location and so that a transmitted RF signal will coalesce with other transmitted RF signals from other RF emitters also traveling through the target location, the signal coalescing action occurring at the target location.

8. The method as described in claim 7 further comprising before said RF emitter firing step, the step of determining target characteristics.

9. The method as described in claim 8 further comprising the step of determining the number of RF emitters to fire at the target location, said emitter number determination being based on one or more determined target characteristics.

10. The method as described in claim 9 further comprising the step of determining the duration of a signal transmission from an RF emitter through the target location, the signal transmission duration determination being related to the target characteristics and the determined number of RF emitters to be fired.

11. The method as described in claim 7 wherein said identified target location determination is accomplished using magnetic resonance imaging techniques.

12. The method as described in claim 7 wherein said identified target location determination is accomplished using computed tomography techniques.

13. The method as described in claim 7 further comprising before said RF emitter firing step, the step of positioning a reflection surface in relation to a patient such that when RF signals are transmitted from RF emitters, the transmitted RF signals will reflect off of the surface of the reflection surface at angles that will cause the reflected RF signals to coalesce at the target location.

14. The method as described in claim 7 wherein said RF emitters orienting step further comprises the steps of:
gathering identified target location information;
determining a location of an RF emitter with reference to the identified target location; and
orienting the RF emitter such that an RF signal transmitted from the emitter will travel through the target location.

15. The method as described in claim 7 wherein said target determination location identification step further comprises identifying the target location by a set of coordinates.

16. The method as described in claim 15 wherein said RF emitters orienting step further comprises the steps of:
gathering coordinates of target location;
calculating an RF emitter location with reference to the identified target location; and
calculating emitter orientation such that a signal transmitted from an RF emitter will travel through the target location.

17. The method as described in claim 16 wherein said RF emitter firing step further comprises establishing a firing sequence such that fired RF signals will coalesce at the identified target location and thereby generate heat at the target location, the heat resulting from the coalescing of the emitted RF signals.

18. The method as described in claim 15 wherein said RF emitters orienting step further comprises the steps of:
calculating coordinates of a target location;
establishing the target location in relation of the predetermined reference location;
calculating the location of an RF emitter in relation to the target location based on a relationship of the RF emitter to the established predefined reference location; and
orienting the RF emitter in relation to the target location.
